(19) United States
(12) Patent Application Publication
(10) Pub. No.: US 2014/0024066 A1
(43) Pub. Date: Jan. 23, 2014

(54) ANIMAL MODEL HAVING PHOTO-ACTIVATABLE MITOCHONDRIA

(71) Applicant: California Institute of Technology, Pasadena, CA (US)

(72) Inventors: David C. Chan, Arcadia, CA (US); Anh H. Pham, Pasadena, CA (US)

(21) Appl. No.: 13/923,218

(22) Filed: Jun. 20, 2013

Related U.S. Application Data
(60) Provisional application No. 61/661,916, filed on Jun. 20, 2012.
(60) Provisional application No. 61/680,160, filed on Aug. 6, 2012.

(51) Int. Cl. G01N 33/50; A01K 67/027

(52) U.S. Cl. CPC G01N 33/5079; A01K 67/0275
USPC 435/29; 800/18

(57) ABSTRACT

An animal model includes a photo-activatable fluorescent protein that can conditionally label the mitochondria throughout the animal or in selective animal cells and tissues, allowing for effective studying of the mitochondria and tracking of mitochondrial dynamics.

### CROSS-REFERENCE TO RELATED APPLICATION(S)

The present application claims priority to and the benefit of U.S. Provisional Application Ser. No. 61/661,916 filed Jun. 20, 2012, and U.S. Provisional Application Ser. No. 61/680,160 filed on Aug. 6, 2012, the entire contents of both of which are incorporated herein by reference.

### STATEMENT REGARDING FEDERALLY SPONSORED RESEARCH OR DEVELOPMENT

This invention was made with government support under R01 GM062967 awarded by the National Institutes of Health. The government has curtain rights in the invention.

### FIELD

This disclosure is directed to an animal model having a mitochondria labeled with a photo-activatable fluorescent protein for studying mitochondrial dynamics.

### BACKGROUND

In recent years, the dynamic properties of mitochondria have become increasingly appreciated. Mitochondria are dynamic and mobile organelles that continually undergo fusion and fission (division). These opposing processes control the morphology of mitochondria, and more importantly, also regulate their physiological functions. As a result, mitochondrial fusion and fission impact cellular respiration, apoptosis, necrosis, and maintenance of mitochondrial DNA. Multiple neurodegenerative diseases have also been associated with defects in mitochondrial dynamics.

Most studies of mitochondrial dynamics rely on cultured cells, in which mitochondria are imaged at high resolution. However, there is a need to extend such studies to tissues, particularly where cell-based models are inadequate in recapitulating complex cellular interactions. It is important to be able to study a broad range of tissues, given that mitochondrial dynamics has been shown to affect the physiology of multiple systems, including the placenta, central nervous system, peripheral nervous system, skeletal muscle, and cardiac muscle (Alexander et al., 2000, Nat Genet. 26: 211-215; Chen et al., 2003, J Cell Biol 160: 189-200; Chen et al, 2007, Cell 130: 548-562; Chen et al., 2010, Cell 141: 280-289; Delettre et al., 2000, Nat Genet. 26: 207-210; Ishihara et 2009, Nat Cell Biol. 11: 958-966; Wakabayashi et al., 2009, J Cell Biol 186: 805-816; Waterham et 2007, N Engl J Med 356: 1736-1741; Zuchner et al., 2004, Nat Genet. 36: 449-451, the entire contents of all of which are herein incorporated by reference). Moreover, the metabolism of tissues can change during developmental transitions, and methods are needed to track mitochondria during such processes. Accordingly, there is a need for studying the mitochondria in vivo in live animal tissues.

### SUMMARY

In some embodiments of the present invention, an animal model includes a first nucleic acid sequence that encodes for a photo-activatable fluorescent protein that targets to a mitochondria in the animal model. The animal model may further include a second nucleic acid sequence configured to prevent expression of the photo-activatable fluorescent protein. The animal model may also include an exogenous recombinase configured to remove the second nucleic acid sequence. The animal model may also include a third nucleic acid sequence defining a recombination site at which the recombinase is configured to excise the second nucleic acid sequence.

In some embodiments, the animal model may be a mouse model. The mouse model may have a first nucleic acid sequence encoding for a photo-activatable fluorescent protein that is capable of being ubiquitously expressed or selectively expressed.

In some embodiments, a method of studying the mitochondria includes studying the mitochondria in an animal model having a first nucleic acid sequence that encodes for a photo-activatable fluorescent protein targeted to the mitochondria.

### BRIEF DESCRIPTION OF THE DRAWINGS

The patent or application file contains at least one drawing executed in color. Copies of this patent or patent application publication with color drawings will be provided by the Office upon request and payment of the necessary fee.

These and other features and advantages of the present invention will be better understood by reference to the following detailed description when considered in conjunction with the accompanying drawings.

FIG. 1A is a schematic showing a strategy for expressing mito-Dendra2 in mice, according to embodiments of the present invention.

FIG. 1B shows a Southern blot analysis gel confirming the insertion of the mito-Dendra2 (6.8 kilobases (kb)) cassette into the Rosa26 gene locus using the probe as indicated in FIG. 1A, according to embodiments of the present invention.

FIG. 1C shows an electrophoresis gel of PCR-amplified DNA from wildtype (wt/wt), wt/PHAM^{floxed} and PhAM^{floxed}/PhAM^{floxed} strains, according to embodiments of the present invention.

FIG. 1D shows an electrophoresis gel of PCR-amplified DNA from PhAM^{floxed}/PhAM^{floxed}, PhAM^{floxed}/PhAM^{excised} and PhAM^{excised}/PhAM^{excised} strains, according to embodiments of the present invention.

FIGS. 2A-2F are images of mitochondria in tail fibroblasts cultured from PhAM^{floxed} mice, according to embodiments of the present invention.

FIG. 3 is a table of data from analysis of mitochondrial morphology of fibroblasts isolated from wildtype (WT), wildtype expressing Cre (WT+Cre), PHAM^{floxed} (PhaM) and PHAM^{floxed} expressing Cre (PhAM+Cre) mouse strains, according to embodiments of the present invention.

FIGS. 4A-4C show time-lapse images of a subset of mitochondria that was photo converted (red), allowing for monitoring of mitochondrial fusion and indicated by the arrowheads, according to embodiments of the present invention.

FIGS. 4D-4F show fluorescence line analysis of the two mitochondria undergoing fusion in FIGS. 4A-4C, according to embodiments of the present invention.

FIG. 5A shows fluorescence from ubiquitous expression of mito-Dendra2 in PHAM^{excised} mouse tissues isolated from pyramidal neurons in the cortex, according to embodiments of the present invention.

FIG. 5B shows fluorescence from ubiquitous expression of mito-Dendra2 in PHAM^{excised} mouse tissues isolated from pyramidal neurons in the hippocampus, according to embodiments of the present invention.

FIG. 5C shows fluorescence from ubiquitous expression of mito-Dendra2 in PHAM^{excised} mouse tissues isolated from Purkinje neurons of the cerebellum, according to embodiments of the present invention.

FIG. 5D shows fluorescence from ubiquitous expression of mito-Dendra2 in PHAM^{excised} mouse tissues isolated from myocardium, according to embodiments of the present invention.

FIG. 5E shows fluorescence from ubiquitous expression of mito-Dendra2 in PHAM^{excised} mouse tissues isolated from testis, according to embodiments of the present invention.

FIG. 5F shows fluorescence from ubiquitous expression of mito-Dendra2 in PHAM^{excised} mouse tissues isolated from lung, according to embodiments of the present invention.

FIG. 5G shows fluorescence from ubiquitous expression of mito-Dendra2 in PHAM^{excised} mouse tissues isolated from liver cannula; the inset shows magnified image of the boxed region, according to embodiments of the present invention.

FIG. 5H shows fluorescence from ubiquitous expression of mito-Dendra2 in PHAM^{excised} mouse tissues isolated from kidney cortex, according to embodiments of the present invention.

FIG. 5I shows fluorescence from ubiquitous expression of mito-Dendra2 in PHAM^{excised} mouse tissues isolated from thymus, according to embodiments of the present invention.

FIG. 6 shows fluorescent images of mito-Dendra2 in PHAM^{excised} mouse tissue sections, in which A-H show mito-Dendra2 signal at low magnification; Ai-Hi depict the respective tissues with merged signal from mito-Dendra2 (green) and cell counterstains (red and blue); Aii-Hii show magnified images of the boxed region; and throughout the tissues sections include: (A) cortex; (B) hippocampus; (C)cerebellum; (D) myocardium; (E) testis; (F) lung; (G) liver; and (H) kidney, according to embodiments of the present invention.

FIG. 7A shows fluorescence of mito-Dendra2 in live spermatocyte cells, according to embodiments of the present invention.

FIG. 7B shows fluorescence of mito-Dendra2 in live myofiber cells, according to embodiments of the present invention.

FIG. 7C shows fluorescence of mito-Dendra2 in live cardiomyocyte cells, according to embodiments of the present invention.

FIGS. 7D-7G are fluorescent images showing a comparison of mito-Dendra2 (green) (FIG. 7D) in a fixed myofiber with the Z-disc marker α-actinin (red) (FIG. 7E), with FIG. 7F showing a merge of FIGS. 7D and 7E; and FIG. 7G is a magnified view of FIG. 7F, according to embodiments of the present invention.

FIGS. 7H-7J are fluorescent images of a subset of mitochondria isolated from EDL muscle that were photo-converted and tracked over a 12-minute period, as indicated, according to embodiments of the present invention.

FIGS. 7K-7N are fluorescent images showing mitochondrial structure during postnatal muscle development; whole EDL muscles were isolated and imaged by mito-Dendra2 fluorescence at indicated ages (FIG. 7K and FIG. 7N), according to embodiments of the present invention; and ultrastructural analysis of fixed EDL sections are shown in FIG. 7L and FIG. 7M, with the mitochondria indicated by arrowheads, according to embodiments of the present invention.

FIG. 8A is a differential interference contrast (DIC) image of subsets of mitochondria in cardiomyocytes that were photo-switched (red), according to embodiments of the present invention.

FIG. 8B is a fluorescent image of subsets of mitochondria in cardiomyocytes that were photo-switched (red), according to embodiments of the present invention.

FIG. 8C is a DIC image of subsets of mitochondria in an EDL myofiber that were photo-switched (red), according to embodiments of the present invention.

FIG. 8D is a fluorescent image of subsets of mitochondria in an EDL myofiber that were photo-switched (red), according to embodiments of the present invention.

FIG. 9A is a merged image of the fluorescence of Purkinje-specific labeling of mitochondria with mito-Dendra2 (green) and anti-calbindin (red), according to embodiments of the present invention.

FIG. 9B is a single-channel image of the anti-calbindin, highlighting the borders of Purkinje neurons of FIG. 9A, according to embodiments of the present invention.

FIG. 9C is a single-channel image of mito-Dendra2, according to embodiments of the present invention.

FIG. 9D is a higher magnification image of the boxed region shown in FIG. 9C, according to embodiments of the present invention.

### DETAILED DESCRIPTION

Embodiments of the present invention are directed toward an animal model having a mitochondria having a photo-activatable fluorescent protein label. Using a photo-activatable fluorescent protein targeted to the mitochondria, the mitochondria may be monitored or “tracked” in live tissues of any established animal model for which techniques exist for genetic modification.

In some embodiments, any photo-activatable fluorescent label may be used that fluoresces and has a modified fluorescence in the presence of a particular wavelength of light. Photoactivatable fluorescent proteins (PMFPs) are known in the art. Non-limiting examples of PAFPs, include Dendra2, Dronpa, Padaron, Kindling FPs, mTFP0.7, rsCherry, and rsCherryRev, all of which are available and described in Chudakov et at, 2010, Physiol. Rev., 90:1103-1163, the entire contents of which are herein incorporated by reference. Additionally, a suitable PAFP is one that is capable of being targeted to the mitochondria. Targeting to the mitochondria may include joining the PAFP sequence to a mitochondria targeting sequence for expression of mitochondrial-targeted photo-activatable fluorescent fusion protein—i.e., mito-PAFP. In this way, non-limiting examples of mito-PAFPs according to embodiments of this invention include mito-Dendra2, mito-Dronpa, mito-Padaron, mito-Kindling FPs, mito-mtTFP0.7, mito-rsCherry; and mito-rsCherryRev.

In some embodiments, an animal model has a mito-PAFP sequence inserted into its genome which can be ubiquitously expressed (expressed in all cells) within the animal model, or selectively expressed in selected cell types within the animal model. In some embodiments, the ubiquitous or selective expression is conditionally expressed with controlled recombination. That is, the nucleic acid sequence encoding the mito-PAFP is combined with at least one upstream termination sequence that prevents expression of the PAFP until the termination sequence is excised. This controlled expression can be regulated using a termination sequence flanked by exogenous recombination sites that are recognized by an exogenous recombinase. For regulated selective expression, the exogenous recombinase that excises the termination sequence is only present in specific cell types.

In some embodiments, a suitable recombination and expression sequence includes at least one regulating sequence (e.g., termination sequence) configured to inhibit expression of the photo-activatable protein sequence. The regulating sequence is the site at which the exogenous recombinase excises the termination sequence to thereby allow for expression of the downstream photo-activatable protein. Suitable recombinase cassettes having recombination site sequences and corresponding recombinases are known in the art. Non-limiting examples of recombinases include Cre, flp and phic 31. Those of ordinary skill in the art can readily utilize any suitable recombination system. In a mouse model, in vivo evaluation of recombinases is established as described in Imayoshi et al., 2012, Neurosci Res 73:106-114 and Birling et al, 2009, Methods Mol. Biol., 561:245-263, the entire contents of both of which are herein incorporated by reference.

In some embodiments, a suitable promoter sequence is used for effective expression and imaging of the mito-PAFP. Non-limiting examples of suitable promoters include the CAG (cytomegalovirus beta-actin) and SV40 (simian vacuolating virus 40) promoters.

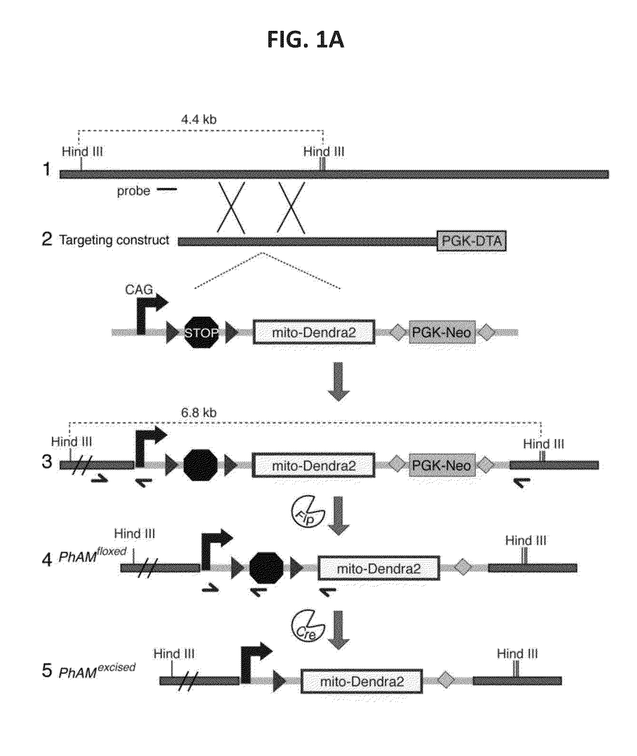

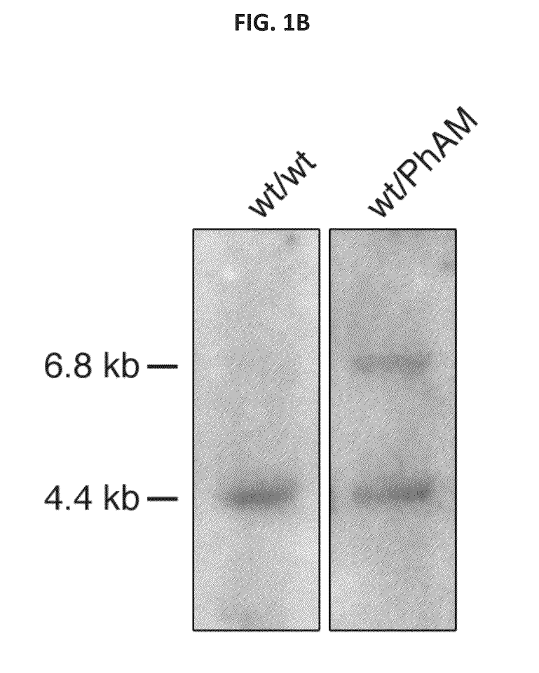

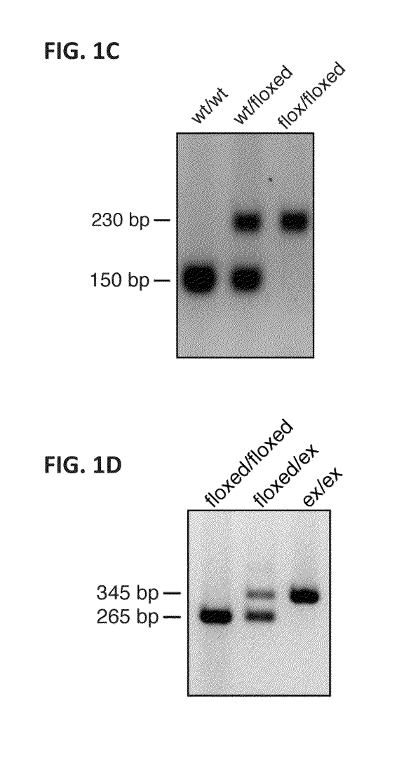

In some embodiments, a mouse model conditionally expresses a mito-PAFP. For example, a mouse line was created from embryonic stem cells transformed with an expression cassette containing the photo-activatable fluorescent protein Dendra2 targeted to the mitochondria (mito-Dendra2). As depicted schematically in FIG. 1A, the expression cassette includes the CAG (CMV/β-actin) promoter and loxP-flanked (foxed) termination sequence upstream of mito-Dendra2 allowing for Cre-regulated expression. Correctly targeted embryonic stem cells were identified as described herein and as shown in FIGS. 1B-1D. Mice having photo-activatable mitochondria were generated using the correctly targeted embryonic stem cells. The strain of mice as described in more detail herein, are referred to as Gt(ROSA)26Sortm1(mito-Dendra2)Dcc, or as the PHAM^{floxed} mouse/mice.

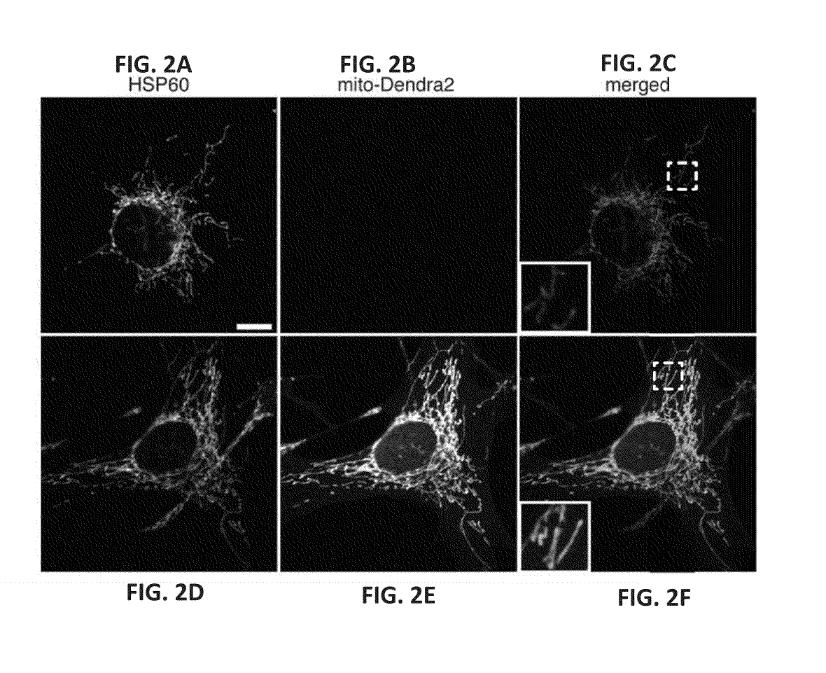

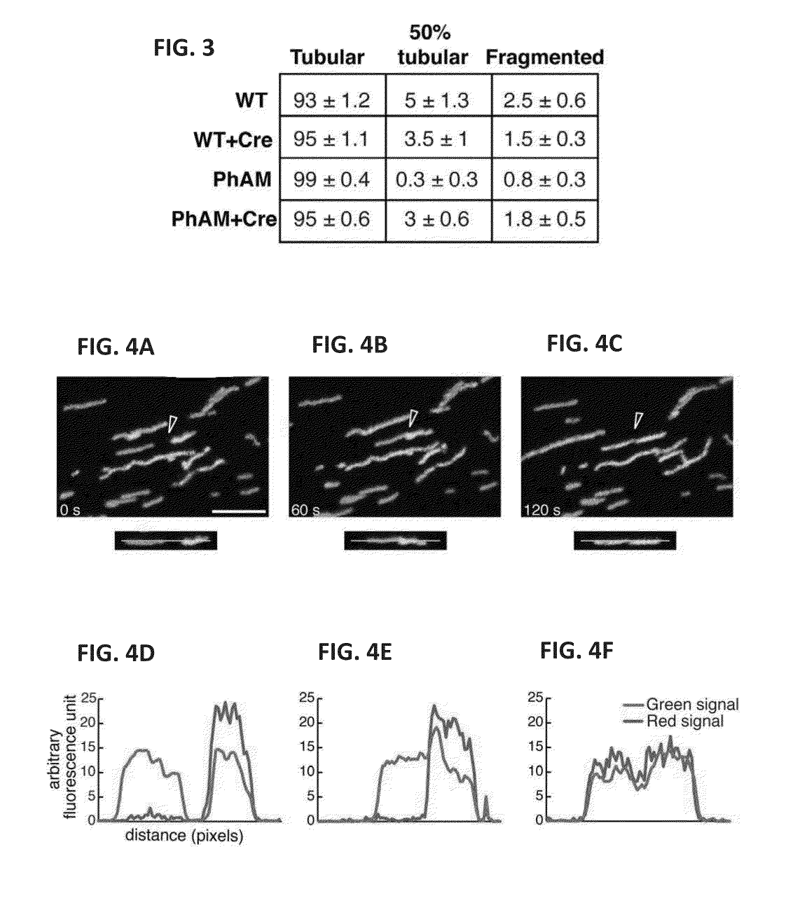

Because the mito-Dendra2 expression is Cre-mediated in the PHAM^{floxed} mouse, no Dendra2 fluorescence is detected in cells isolated from this mouse, as shown in FIGS. 2A, 2B, and 2C. However, in the presence of Cre recombinase, the mito-Dendra2 protein is expressed, and subsequently, the cells show bright green fluorescence which co-localizes with HSP-60, a marker protein of the mitochondrial matrix, as shown in FIGS. 2D, 2E, and 2F. Furthermore, FIG. 3 shows data from mitochondrial morphology analysis of wildtype (WT), (WT and Cre), (PhAM), and (PhAM and Cre) fibroblasts, which indicate that the expression of mito-Dendra2 does not alter the morphology of the mitochondrial network, and as such, the photo-activatable mitochondria is not altered for subsequent tracking in the PHAM^{floxed} mouse.

In some embodiments, the PHAM^{floxed} mouse line can be combined with Cre drivers to restrict mito-PAFP expression to specific cells, facilitating the analysis of mitochondria in tissues with high cell diversity. For example, many cell- and tissue-specific Cre-lox recombinase expression systems are available from The Jackson Laboratory, Bar Harbor, Me., thereby allowing for expression of the mito-PAFP in selected cell types.

In some embodiments, a method of tracking mitochondrial dynamics includes photo-activating a population of mitochondria in live cells. That is, using a laser directed at mito-Dendra2 cells, the mito-Dendra2 protein receiving the 405 nm wavelength of light from the laser is converted from the green fluorescence to red fluorescence, thereby effecting a red labeling of a sub-population of mitochondria within a larger population of green-labeled mitochondria for effective tracking of the red-labeled mitochondria. As an example, a time-lapsed video was captured of photo-converted mitochondria in fibroblasts isolated from a PHAM^{floxed} mouse. The video shows both transport and fusion of these labeled mitochondria where the fusion events between the red and green mitochondria result in the transfer of fluorescence signal (yellow), indicating mitochondrial matrix mixing (FIGS. 4A-4F).

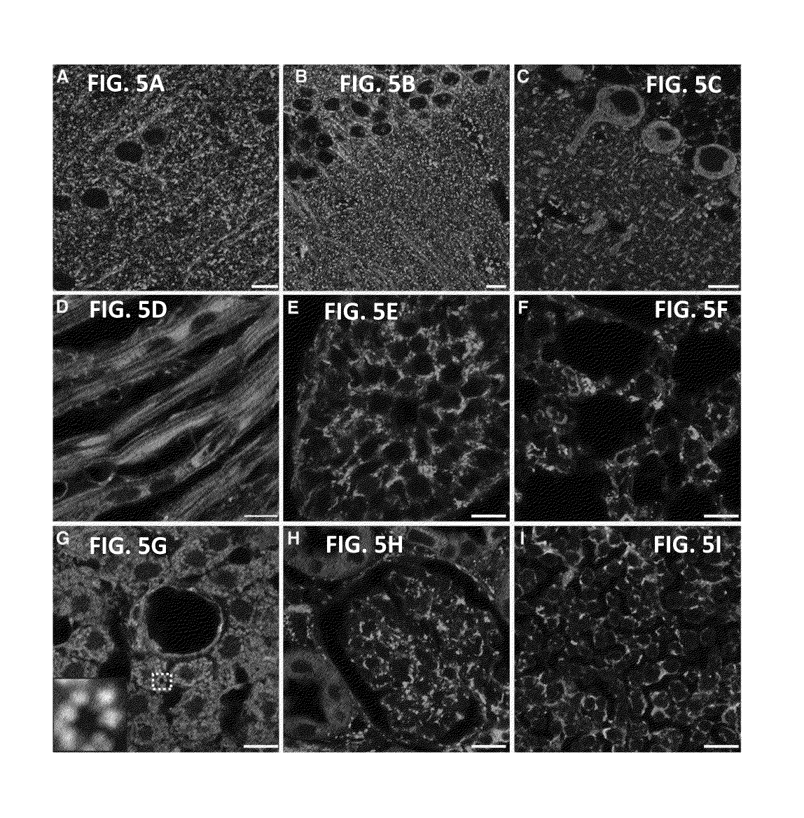

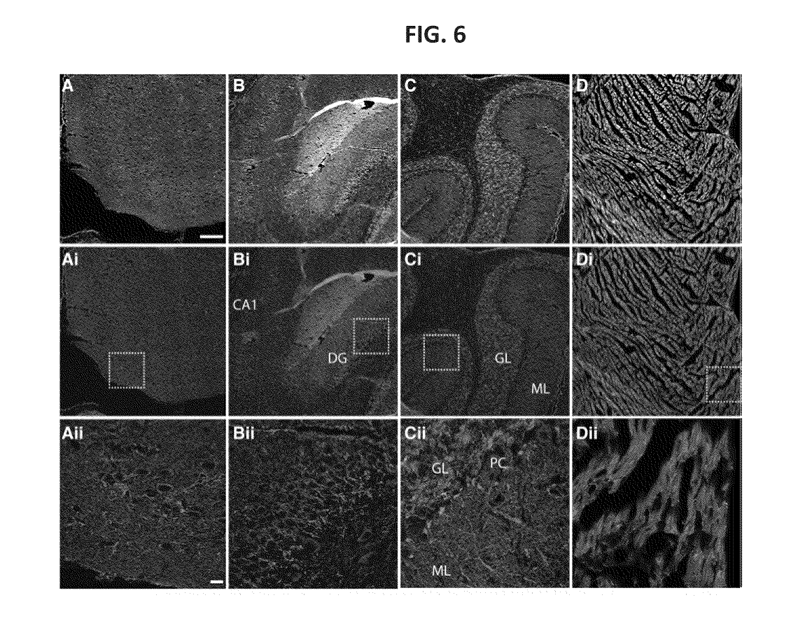

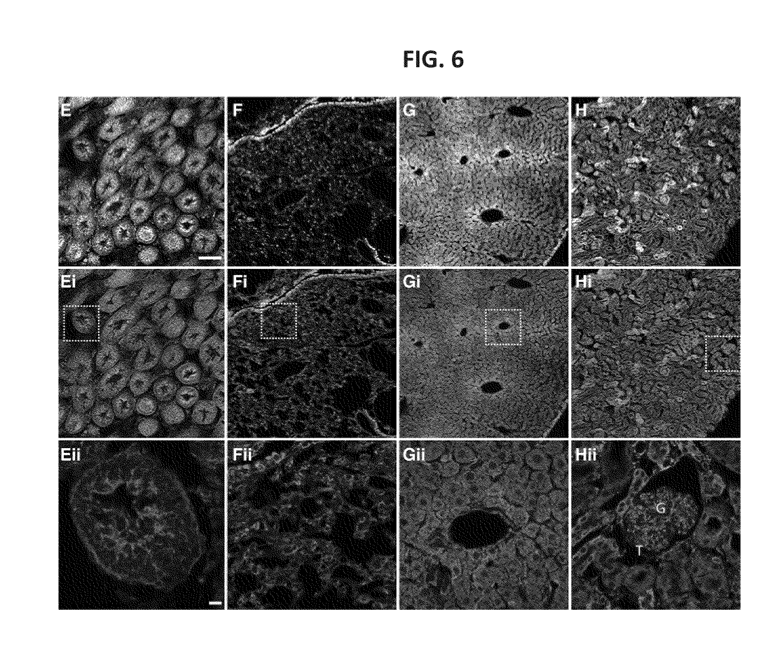

In some embodiments, mitochondrial morphology is monitored in an array of tissues in an animal model having ubiquitous expression of a photo-activatable fluorescent protein targeted to mitochondria throughout the cells of the animal. For example, a mouse line was generated having ubiquitous expression of mito-Dendra2. Specifically, a mouse lacking the flexed termination cassette was isolated from crossing the PHAM^{floxed} mouse with a Meox2-Cre mouse. This mouse is referred to as PHAM^{excised} (FIGS. 1A and 1D). In tissue sections, all organs isolated from PHAM^{excised} mice exhibit bright mito-Dendra2 fluorescence localized specifically to the mitochondrial compartment. Widespread expression of mito-Dendra2 is found in the central nervous system, heart, testis, lung, liver, kidney, and thymus (FIGS. 5A-5I and FIG. 6). As such, the PHAM^{excised} line can be used to survey mitochondrial morphology in a wide range of tissues. For example, cardiomyocytes contain linearly aligned mitochondria in contrast to the punctate structures found in hepatocytes (compare FIG. 5D to FIG. 5G), Furthermore, homozygous PHAM^{excised} mice are viable and fertile.

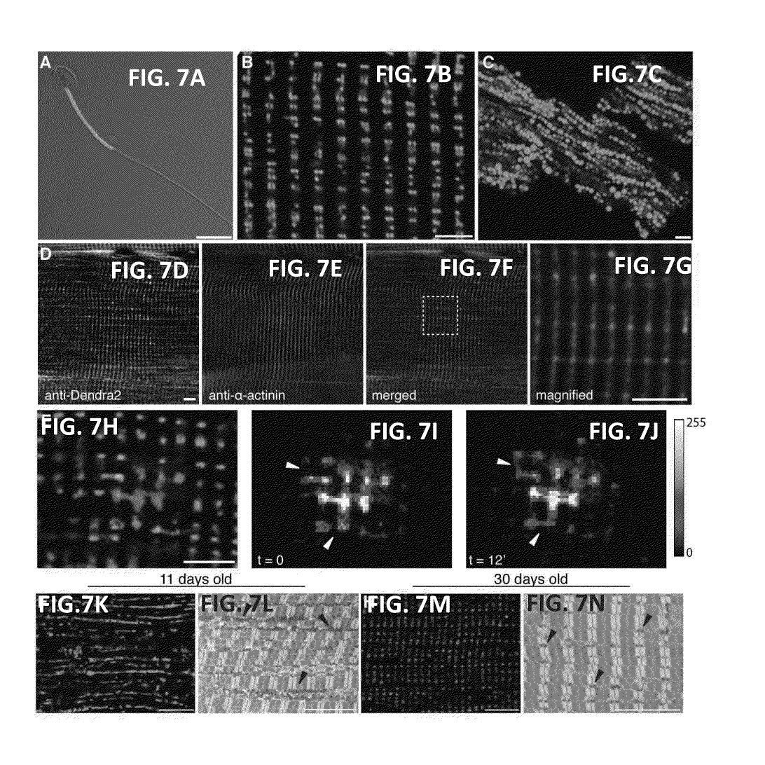

In some embodiments, live cells are isolated from an animal model having mitochondria labeled with a photo-activatable fluorescent protein to facilitate imaging of the mitochondria network. For example, live cells may be isolated from various cell types in PHAM^{excised} mice as shown in FIGS. 7A through 7N. In live mouse sperm (FIG. 7A), a region of intense mito-Dendra2 fluorescence was observed in the midpiece of the sperm. Individual mitochondria were not resolved in the midpiece, suggesting that these mitochondria are packed tightly. When a small portion of the midpiece was illuminated with the 405 nm laser as indicated by the red fluorescence in FIG. 7A, the photo-converted region was found to be stable, indicating that the packed mitochondria are discrete and do not share matrix contents.

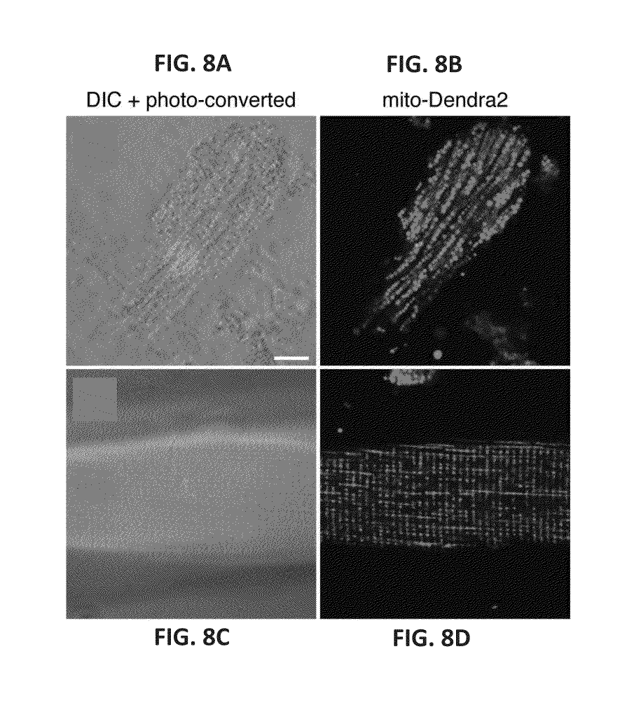

In some embodiments, mitochondria in dissociated tissues and intact skeletal muscles isolated from a PHAM^{excised} mouse are labeled with mito-Dendra2 (FIG. 7B). In collagenase digested myofibers, mito-Dendra2 fluorescence is arranged in a repeating pattern of doublets (FIG. 7B, and FIGS. 8A-8D). In fixed myofibers, mito-Dendra2 signal localizes adjacent to the Z-disk marker, α-actinin (FIG. 7D). This pattern is consistent with ultra-structural studies showing the stereotyped architecture of mitochondria in skeletal muscle. In dissociated cardiomyocytes isolated from a PHAM^{excised} mouse, mitochondria are arranged in linear arrays (FIG. 7C and FIGS. 8A-8D). in each case, the photo-conversion of Dendra2 provides higher resolution of mitochondria in dense networks (FIGS. 8A-8D).

In some embodiments, mitochondria are tracked in live tissues of a photoactivatably labeled animal model. For example, mitochondria may be tracked in live tissues from a PHAM^{excised} mouse. Mitochondrial dynamics in whole extensor digitorum longus (EDL) muscles in a PHAM^{excised} mouse were monitored. By following a subset of photo-converted mitochondria over time, mitochondrial fusion between intramyofibrillar mitochondria was observed. The fusion events occurred along both the longitudinal and transverse axes of the myofiber (FIG. 7E). Therefore, although mitochondria in skeletal muscle appear static and rigidly organized, they are dynamic and fusion-competent.

It was previously observed that postnatal development of fast-twitch muscle is accompanied by a dramatic increase in mitochondrial DNA copy number (Chen et al, 2010, supra). This observation suggests that mitochondria may play a role in the development of skeletal muscle. In some embodiments, mitochondrial morphology is studied during the postnatal development of EDL muscle in a PHAM^{excised} mouse. In fixed whole mounts of EDL, a dramatic remodeling of mitochondrial structure between postnatal day 11 and 30 was observed (FIG. 7F-7H). In EDL muscle at postnatal day 11, the mitochondria appear as elongated tubules oriented along the long axis of the muscle fiber (FIG. 4F). By postnatal day 30, the mitochondria are punctate and organized into doublets (FIG. 4H). These morphological observations in the PHAM^{excised} muscles were confirmed by electron microscopy analysis of wildtype mice (FIGS. 7G and 7I). Taken together, these results show that the PhAM mouse lines disclosed herein can be used to examine mitochondria in developmental processes in a variety of cell types.

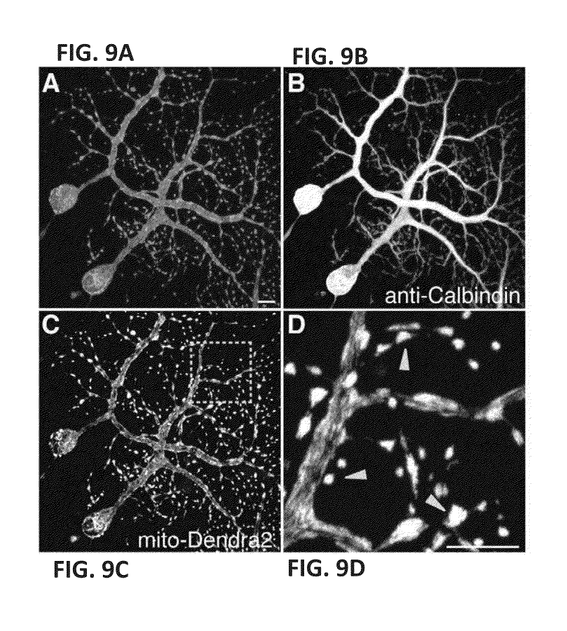

In some embodiments, an animal model has a photo-activatable label expressed in the mitochondria of one cell type. For example, in the PHAM^{excised} mouse, the near ubiquitous expression of mito-Dendra2 in some tissues with high cell density and diversity may cause overlapping mitochondrial signals from multiple cells. As such, expressing mito-Dendra2 in a particular cell type or selected cell types, avoids overlapping signals from multiple cell types. Pcp2-Cre mice express Cre exclusively in Purkinje neurons of the cerebellum. As such, PHAM^{floxed} mice were crossed with the Pep2-Cre mice, generating mice having mito-Dendra2 expressed in Purkinje neurons of the cerebellum. To facilitate high resolution imaging in brain tissue, organotypic culturing methods were used to maintain parasagittal cerebellar slices. Purkinje neurons in the cerebellum were identified by anti-calbindin immunofluorescence. As expected, mito-Dendra2 expression is restricted to Purkinje neurons (FIGS. 9A-9C). In these neurons, several morphologically distinct populations of mitochondria were observed (FIG. 9D).

The PHAM^{excised} and PHAM^{floxed} mouse models as disclosed herein allow for tracking of mitochondria dynamics in mouse tissues and cells. Additionally, the photo-activatable mito-Dendra2 enables high-resolution analysis and direct measurement of mitochondrial fusion in live cells.

The following Examples are presented for illustrative purposes only, and do not limit the scope or content of the present application.

### EXAMPLES

Reference is made to Pham et al, 2012, Genesis, 50:833-843, the entire contents of which is incorporated herein by reference.

### Example 1

### Construction of the Gt(ROSA)26Sortm1(mito-Dendra2)Dcc (PHAM^{floxed}) Mouse Line

The mito-Dendra2 expression cassette was assembled in a modified pBluescript shuttle plasmid (kindly provided by Dr. John Burnett). Dendra2 was purchased from Evrogen and mito-GFP (pAcGFP1-Mito Vector) was purchased from Clontech. The CAG enhancer-promoter was transferred from Rosa26 mT/mG (Muzumdar et al., 2007, Genesis 45: 593-605, the entire contents of which is herein incorporated by reference) and the floxed termination signal derived from pBS302 (Sauer, 1993, Methods Enzymol 225: 890-900, the entire contents of which is herein incorporated by reference). This floxed termination signal is composed of two loxP sites flanking the SV40 polyadenylation signal sequence. To localize Dendra2 (Chudakov et al., 2007, Nat Protoc 2: 2024-2032, the entire contents of which is herein incorporated by reference) to the mitochondria, the N-terminus was fused to the mitochondrial targeting sequence of subunit VIII of cytochrome c oxidase. The bovine growth hormone (bGH) polyadenylation signal was placed downstream of Dendra2. All plasmids were verified by DNA sequence analysis.

The mito-Dendra2 targeting construct was linearized with PvuI and electroporated into low passage 129/SvEV ES cells as previously described (Chen et al., 2003, supra). Of 94 neomycin-resistant clones, four were correctly targeted, as determined by PCR and Southern blot analysis. One ES clone was injected into C57BL/6 blastocysts to generate chimeric mice. Founder chimeric mice were bred to C57BL/6 to confirm germline transmission. The mice were then crossed with FLPeR mice to remove the neomycin-resistance cassette, thereby generating the PHAM^{floxed} line. (Farley et at 2000, Genesis 28: 106-110, the entire contents of which is herein incorporated by reference.) The PHAM^{floxed} mice were crossed with the Meox2-Cre mice (Tallquist and Soriano, 2000, Genesis 26: 113-115, the entire contents of which is herein incorporate by reference) to generate the PHAM^{excised} line.

As shown schematically in FIG. 1A, mito-Dendra2 was targeted to the Rosa26 gene locus. Schematic 1 represents the wild-type Rosa26 locus with a 4.4 kilobase (kb) HindIII region as shown. The targeting construct (Schematic 2) contains a floxed termination signal upstream of mito-Dendra2 and flanking sequences from the Rosa26 genomic DNA, followed by a PGK-DTA (phosphoglycerate kinase-diptheria toxin gene) cassette (derived from Rosa26 mT/mG purchased from Addgene) having a neomycin (Neo) selection marker. Homologous recombination in embryonic stem cells results in the insertion of the construct into the Rosa26 locus (Schematic 3). In mice, removal of the neomycin selection marker by Flp recombinase results in the PHAM^{floxed} line (Schematic 4), which can be mated to a Cre driver line to obtain cell-specific labeling of mitochondria. Germline excision of the termination signal produced the PHAM^{excised} line (Schematic 5). Black arrowheads, loxP sites; stop symbol, termination cassette; gray diamonds, flippase recognition target (frt) sites; half arrows, PCR primers for genotyping; short horizontal line, probe for Southern blot.

The mito-Dendra2 expression relies on Cre-mediated excision of the termination sequence, and as such, PHAM^{floxed} mice may be maintained as heterozygotes or homozygotes without apparent defects in viability or fertility.

### Example 2

### Confirmation of the PHAM^{floxed} and PHAM^{excised} Alleles

For Southern analysis, genomic DNAs were digested with HindIII and hybridized with the published probe from the pROSA26-5′ plasmid (Soriano, 1999, Nat Genet. 21: 70-71). To genotype the PHAM^{floxed} allele by PCR, the set of three primers were used: Rosa4 5′-TCAATGGGCGGGGGTCGTT (SEQ ID NO: 1) (Zong et al., 2005, Cell 121: 479-492, the entire contents of which is herein incorporated by reference). R26-F 5′-TCCTGGCTTCTGAGGACCGC, (SEQ ID NO: 2) and R26-R 5′-TTCCCCTGCAGGACAACGCC, (SEQ ID NO: 3). The wild-type allele yields a 150 bp band while the mito-Dendra2 insertion results in a 252 bp band. Germline excision of the termination sequence was verified using the following set of primers: CAG 5′-TACAGCTCCTGGGCAACGTGCT, (SEQ ID NO: 4), Stop 5′-TGGCAGCAGATCTAACGGCCG, (SEQ ID NO: 5), Dendra2 5′-GTTCACGTTGCCCTCCATGT, (SEQ ID NO: 6). The lower 265 bp band is derived from the termination cassette whereas the upper 345 bp band represents Cre-mediated excision of the foxed region.

FIG. 1B is a representative Southern blot analysis of ES cell clones. Genomic DNA was digested with HindIII and hybridized with the Rosa26 probe indicated in schematic 1 of FIG. 1A. FIG. 1C is an electrophoresis gel from PCR genotyping of the PHAM^{floxed} strain for the wild-type or knock-in allele using the set of three primers in Schematic 3 of FIG. 1A. FIG. 1D is an electrophoresis gel from PCR genotyping of the PHAM^{excised} strain using the three primers in Schematic 4 of FIG. 1A.

### Example 3

### Antibodies and Cell Stains

The following dyes were used: wheat germ agglutinin A594 (1:250, Molecular Probes), NeuroTrace fluorescent Nissl stain A640 (1:200, Molecular Probes), DAPI (300 nM, Molecular Probes), and Alexa Fluor 546 streptavidin (1:500, Molecular Probes). Primary antibodies included: mouse anti-Map2 (1:1000, Sigma), mouse anti-calbindin (1:1500, Sigma), goat anti-HSP60 (1:200, Santa Cruz), rabbit anti-Dendra2 (1:500, Evrogen), and mouse anti-α-actinin (1:100, Sigma). Secondary antibodies included biotinylated goat anti-mouse (Vector labs), Alexa Fluor 546 donkey anti-goat, Alexa Fluor 546 donkey anti-mouse, and Alexa Fluor 488 goat-anti-rabbit (1:500, Molecular probes).

### Example 4

### Histological Analysis

For all histological sections, mice were perfused transcardially with phosphate buffered saline (PBS) followed by 10% formalin (Sigma). Tissues were embedded overnight at 4° C. in 30% sucrose solution and frozen in OCT (optimal cutting temperature) for sectioning by a cryostat. For fluorescence staining, slides were either incubated with primary antibodies overnight or overlaid with WGA (wheat germ agglutinin) or Nissl stain for 1-2 hours at room temperature.

For organotypic slices, membranes surrounding the cerebellum were trimmed and fixed overnight with 4% paraformaldehyde-lysine-periodate fixative at 4° C. Slices were permeabilized with 1% Triton-X for 15 minutes and incubated with blocking buffer (2% goat serum, 1% BSA, and 0.1% Triton-X) for 4-6 hours. Samples were incubated with primary antibodies overnight followed by secondary antibodies for 2 hours.

To stain muscles, EDL samples were fixed for 1 hour at room temperature with 10% formalin. Myofibers were mechanically teased apart and immunostained with the Vector M.O.M. kit (Vector labs) according to the manufacturer's protocol.

### Example 5

### Fibroblast Cells

Tail fibroblasts were isolated from the wildtype or PHAM^{floxed} line by trypsin-EDTA digestion of skin fragments from the tail. After several days in culture with media containing DMEM, 10% fetal bovine serum, 1 mM L-glutamine, and 1× penicillin/streptomycin (Life Technologies/GIBCO), fibroblasts from hair follicles migrated onto the plate. To facilitate immortalization, these fibroblasts were transduced with retrovirus harboring SV40 large T antigen. For assessment of mitochondrial morphology, fibroblasts were plated in 8-well chamber slides. In each well, 200 cells were classified into one of three mitochondrial profiles: 100% tubular, 50% mixture of fragmented and short tubules, or completely fragmented.

FIGS. 2A-2F are representative images of mitochondria in tail fibroblasts cultured from the PHAM^{floxed} mice. Tail fibroblasts were cultured in the absence (FIGS. 2A-2C) or presence of Cre-expressing retrovirus (FIGS. 2D-2F). Mitochondria are identified by immunostaining for HST60 (red). The mito-Dendra2 signal (green) was found only after the expression of Cre. Mitochondrial morphology remains tubular (inset). Scale bar shown is 10 μm. FIG. 3 is a table of data from mitochondrial morphology analysis in wildtype and PHAM^{floxed} fibroblasts. The table shows the percentage of cells with the indicated morphology±SEM (n=4). FIGS. 4A-4C shows images from monitoring of mitochondrial fusion in PHAM^{floxed} fibroblasts. A subset of mitochondria was photo-converted (red) and tracked by time-lapse imaging. Three still images from the resulting movie highlight a mitochondrial fusion event (arrowhead) and exchange of matrix contents. Scale bar shown is 5 μm. FIGS. 4D-4F shows the fluorescence line analysis of the two mitochondria undergoing fusion in the frames of FIGS. 4A-4C. Each plot measures the red and green signals along the drawn line. The line analysis demonstrates that mitochondrial fusion results in the transfer of red fluorescence to the adjoining mitochondrion.

### Example 6

### Isolated Cells & Tissues

To isolate primary cardiomyocytes for live imaging of mitochondrial dynamics, the ventricles were rinsed with cold PBS supplemented with 10 mg/ml of glucose and mechanically minced in 0.5% collagenase PBS buffer. The tissue was digested in 20 min intervals at 37° C. in a rotary shaker, and myocyte supernatants were collected and pooled between digestion intervals until minimal ventricular tissues remained. Only rod shaped ventricular myocytes were selected for imaging.

For primary myofibers, the EDL muscle was digested with 4 mg/ml of collagenase in DMEM media for 1 hr at 37° C. in a rotary shaker. Digested EDL muscles were triturated several times using decreasing bore sizes of flame-heated pasteur pipettes to obtain individual myofibers for live imaging. For whole muscle imaging, the EDL was removed, placed in a coverglass bottom petri dish, and held in place using a slice anchor (Warner instruments). Whole muscles were imaged in media containing DMEM (no phenol red), 10% fetal bovine serum, 1 mM pyruvate, and 25 mM HEPES.

Mouse sperm was isolated from the cauda epididymis of 2-3 months old males. Longitudinal cuts were made along the epididymus to enable motile, mature sperm to swim out into the PBS solution. All live samples were imaged on a stage-top heated platform maintained at 37° C.

FIGS. 5A-5I are images of frozen tissue sections from the PHAM^{excised} mice, as disclosed herein. FIG. 5A is an image of pyramidal neurons in the cortex; FIG. 5B is an image of pyramidal neurons in the hippocampus; FIG. 5C is an image of Purkinje neurons of the cerebellum; FIG. 5D is an image of myocardium; FIG. 5E is an image of testis; FIG. 5F is an image of lung; FIG. 5G is an image of liver cannula, inset shows magnified image of the boxed region; FIG. 5H is an image of kidney cortex; FIG. 5I is an image of thymus. Cell counter stains are shown in red or purple. In FIGS. 5A-5B, anti-Map2 (red) stains the dendritic processes of neurons; in FIGS. 5A-5B, a fluorescent Nissl stain (purple) marks neurons; in FIG. 5C, anti-calbindin (red) highlights Purkinje neurons; in FIGS. 5D-5I wheat germ agglutinin (WGA) labels cell borders. Scale bars shown represent 10 μm.

FIG. 6 shows images of mito-Dendra2 signal at low magnification. Ai-Hi depict the respective tissues with merged signal from mito-Dendra2 (green) and cell counterstains (red and blue.) Aii-Hii show magnified images of the boxed region. (A) cortex; (B) hippocampus; (C) cerebellum; (D) myocardium; (E) testis; (F) lung; (G) liver; (H) kidney. For A-C, counterstains included anti-Map2 (red) to label neuronal dendrites and a fluorescent Nissl stain (blue) to mark neurons; in D-H wheat germ agglutinin (red) delineates cell borders. Scale bar represents 100 μm in low magnification images and 10 μm in the high magnification images.

### Example 7

### Organotypic Slice Cultures

Pups from postnatal days 10-12 were used for organotypic cultures. Tail samples from each animal were retained for genotyping. The cerebellum was removed and incubated in ice-cold preparation media containing 1×GBSS (Sigma) supplemented with 6.5 mg/ml of glucose. Each hemisphere was glued onto a rotating magnetic stage for sectioning by a Leica VT 1200S vibratome. For each animal, approximately 4-6 sections (2-3 per hemisphere) of 330 μm thickness were collected and transferred to a petri dish with cold preparation media using a wide bore pipette. Evenly sliced sections were selected under the dissecting scope and transferred to millicell membrane inserts (Millipore, PICM3050) in a 6 well plate. Typically, 2-4 sections were plated onto one insert for culturing by the interface method at 35° C. with 5% CO₂ (Stoppini et al., 1991, J Neurosci Methods 37: 173-182, the entire contents of which is herein incorporated by reference). The culture medium is a mixture of MEM (Life Technologies, 51200), 2 mM L-glutamine, 1 mM GlutaMAX (Life Technologies, 35050), 0.5 mg/ml penicillin-streptomycin, 50% heat-inactivated horse serum, 25% Hank's salt solution, 10 mM HEPES, and 6.5 mg/ml of glucose. The media was buffered to a pH of 7.2. Slices were fed with new media on alternating days 3 times a week and equilibrated in culture for at least 10 days prior to experimentation.

FIGS. 9A, 9B, 9C, and 9D are images from organotypic slice cultures. PhAM^{floxed} mice were crossed with a Purkinje-specific driver, Pcp2 Cre, and organotypic slice cultures were prepared from the offspring. FIG. 9A is a merged image of mito-Dendra2 (green) and anti-calbindin (red). Two Purkinje cells express mito-Dendra2. FIG. 9B is a single-channel image of anti-calbindin highlighting the borders of Purkinje neurons. FIG. 9C is a single-channel image of mito-Dendra2 signal. FIG. 9D is a high magnification image of the boxed region in FIG. 9C. The tight clusters of mitochondria in the distal dendritic branches are indicated with arrowheads, with the scale bars representing 10 μm.

### Example 8

### Microscopy Analysis

Images were acquired with a Zeiss LW 710 confocal microscope with EC-Plan-Neofluar 40×/1.3 oil and Plan-Apochromat 63×/1.4 oil objectives. Z-stack acquisitions over-sampled each optical slice twice, and the Zen 2009 image analysis software was used for maximum z-projections. The 488 nm laser line and the 561 nm laser excited Dendra2 in the unconverted state and photo-converted state, respectively. To photo-switch Dendra2, a region was illuminated with the 405 nm line (4% laser power) for 90 bleaching iterations at a scan speed of 6.3-12.61 μs/pixel, Alexa 594 and Alexa 640 conjugated dyes were excited by the 561 nm laser and the 633 nm laser, respectively. For live imaging of primary cardiomyocytes, sperm, and myofibers, the C-Apochromat 63×/1.2W objective was used.

The fluorescence of mito-Dendra2 (green) was imaged in a FIG. 7A spermatocyte, FIG. 7B myofiber, and FIG. 7C cardiomyocyte. In each case, a subset of mitochondria was irradiated with a 405 nm laser to photo-switch mito-Dendra2 (red). FIGS. 7D-7G shows a comparison of mito-Dendra2 (green) in a fixed myofiber with the Z-disc marker α-actinin (red). Since the myofiber FIGS. 7D-7G was processed for immunostaining, the resolution of mitochondrial doublets is lower than in FIG. 7B. FIGS. 7H-7J show the images from detection of mitochondrial fusion in isolated EDL muscle from a 2-month old animal. A subset of mitochondria was photo-converted and tracked. Intensity maps (FIGS. 7I and 7J) of the photo-converted signal show two mitochondrial fusion events (marked by arrowheads) over a 12-minute period. In the top fusion event, the transfer of red signal into an unconverted mitochondrion was detected. In the bottom event, fusion occurs between two photo-converted mitochondria and results in equalization of the intensity. Intensity values of the heat maps are indicated in the legend. Scale bars: 10 μm for sperm and 5 μm for myofibers and cardiomyocyte. FIGS. 7K and 7M show images in mitochondria structure during postnatal muscle development. Whole EDL muscles were isolated and imaged by mito-Dendra2 fluorescence at indicated ages with scale bars showing 5 μm. FIGS. 7L and 7N show ultrastructural analysis of fixed EDL sections. Mitochondria are indicated by arrowheads, with scale bars showing 10 μm.

FIGS. 8A-8D show images of subsets of mitochondria that were photo-switched to red fluorescence to enhance the resolution of mitochondria in dense networks. Labeled images of an isolated cardiomyocyte (FIGS. 8A, 8B) and an EDL myofiber (FIGS. 8C, 8D) in which the scale bar represents 10 μm.

As disclosed throughout and evidenced by FIGS. 2F, 4A-4C, 7A-7N, mice expressing mito-Dendra2 are an effective tool for monitoring mitochondria dynamics.

While the present invention has been illustrated and described with reference to certain exemplary embodiments, those of ordinary skill it the art will understand that various modifications and changes may be made to the described embodiments without departing from the spirit and scope of the present invention, as defined in the following claims.

What is claimed is:

1. An animal model, comprising:
a first nucleic acid sequence that encodes for a photo-activatable fluorescent protein that targets to a mitochondria in the animal model.

2. The animal model of claim 1, further comprising a second nucleic acid sequence configured to prevent expression of the photo-activatable fluorescent protein.

3. The animal model of claim 2, further comprising an exogenous recombinase configured to remove the second nucleic acid sequence.

4. The animal of claim 3, further comprising a third nucleic acid sequence defining a recombination site at which the recombinase is configured to excise the second nucleic acid sequence.

5. The animal model of claim 3, wherein the exogenous recombinase is selected from Cre, flippase, and phic 31.

6. The animal model of claim 1, wherein the animal model is a mouse.

7. The animal model of claim 6, wherein the photo-activatable fluorescent protein is selected from mito-Dendra2, mito-Dronpa, mito-Padaron, mito-Kindling FPs, mito-mTFP0.7, mito-rsCherry, and mito-rsCherryRev.

8. The animal model of claim 6, wherein the photo-activatable fluorescent protein is Dendra2.

9. The animal model of claim 8, wherein the Dendra2 is ubiquitously expressed.

10. The animal model of claim 8, wherein the Dendra2 is selectively expressed.

11. The animal model of claim 1, wherein the photo-activatable fluorescent protein is ubiquitously expressed.

12. The animal model of claim 1, wherein the photo-activatable fluorescent protein is selectively expressed.

13. A method of studying mitochondria, comprising studying the mitochondria in the animal model of claim 1.

14. The method of claim 13, wherein the studying the mitochondria in the animal model, comprises isolating tissue from the animal, the tissue comprising the mitochondria in which the photo-activatable fluorescent protein is expressed.

15. The method of claim 14, further comprising photo-activating the photo-activatable fluorescent protein in the mitochondria in which the photo-activatable fluorescent protein is expressed.


SEQUENCE LISTING (6 sequences)

SEQ ID NO: 1 - DNA, 19 bp, Artificial Sequence
  PhAM floxed primer
tcaatgggcgggggtcgtt

SEQ ID NO: 2 - DNA, 20 bp, artificial sequence
  PhAM floxed primer
tcctggcttctgaggaccgc

SEQ ID NO: 3 - DNA, 20 bp, Artificial Sequence
  PhAM floxed primer
ttcccctgcaggacaacgcc

SEQ ID NO: 4 - DNA, 22 bp, Artificial Sequence
  PhAM Termination Primer
tacagctcctgggcaacgtgct

SEQ ID NO: 5 - DNA, 21 bp, Artificial Sequence
  PhAM Termination Primer
tggcagcagatctaacggccg

SEQ ID NO: 6 - DNA, 20 bp, Artificial Sequence
  PhAM Termination Primer
gttcacgttgccctccatgt